(19) United States
(12) Patent Application Publication
(10) Pub. No.: US 2022/0033591 A1
(43) Pub. Date: Feb. 3, 2022

(54) GEL PRECURSOR, NUCLEIC ACID AMPLIFICATION REAGENT GEL, STORAGE CHIP AND USING METHOD THEREOF

(71) Applicant: BOE Technology Group Co., Ltd., Beijing (CN)

(72) Inventor: Weifeng XU, Beijing (CN)

(73) Assignee: BOE Technology Group Co., Ltd.

(21) Appl. No.: 17/198,406

(22) Filed: Mar. 11, 2021

(30) Foreign Application Priority Data
Jul. 29, 2020 (CN) 202010745599.7

(51) Int. Cl. C08J 3/075; C12Q 1/6844

(52) U.S. Cl. CPC C08J 3/075; C12Q 1/6844; C08J 2405/12; C08J 2405/04; C08J 2305/12

(57) ABSTRACT

The present disclosure can provide a gel precursor, a nucleic acid amplification reagent gel, a storage chip and a using method thereof. The gel precursor includes: a sodium alginate main solution mixed with at least one of the following components: starch, dextrin, chitosan, agarose and gelatin, where the sodium alginate main solution has a mass concentration range from 0.01% w/v to 5% w/v.

### CROSS REFERENCE TO RELATED APPLICATIONS

The present disclosure is based on and claims priority under 35 U.S.C. 119 to Chinese Patent Application No. 202010745599.7 filed on Jul. 29, 2020, in the China National Intellectual Property Administration, the entire content of which is incorporated herein by reference.

### FIELD

The present disclosure relates to the field of a preservation method of a biochemical reagent composition, in particular to a gel precursor, a nucleic acid amplification reagent gel, a storage chip and a using method.

### BACKGROUND

In Vitro Diagnosis (IVD) is a product and service to perform detection in vitro of a human body by utilizing biological samples (blood, body fluid, tissue, etc.) to obtain clinic diagnosis information, and then to judge diseases or body functions. The technology of detecting and analyzing information such as nucleic acid chains or nucleic acid segments contained in samples via nucleic acid amplification (e.g., a polymerase chain reaction and a loop-mediated isothermal amplification reaction) belongs to the scope of molecular diagnosis. Duo to capability of diagnosing diseases and obtaining body information at the nucleic acid level, molecular diagnosis is considered as a more precise diagnosis method that can shorten the pre-seroconversion window period, and molecular diagnosis has increasing shares in the field of in vitro diagnosis and has the highest growth rate. There are many types of reagents required for molecular diagnosis, and a method of manual step-by-step adding for operation is usually adopted in a laboratory, which is relatively tedious and prone to bringing pollution and must be operated by specialists. With the expansion of application fields and market of molecular diagnosis, the demand for portable and one-step-operation small-sized point of care testing (POCT) equipment is also increasing, which requires increasing the integration of equipment and diagnosis consumables, and one of development trends is to pre-store reagents needed for completing all reactions in the equipment or diagnosis consumables in advance so as to use the reagents anytime.

### SUMMARY

The present disclosure provides a gel precursor, a nucleic acid amplification reagent gel, a storage chip and a using method thereof. The gel precursor includes: a sodium alginate main solution mixed with at least one of the following components:

starch;

dextrin;

chitosan;

agarose; and

gelatin;

where the sodium alginate main solution has a mass concentration range from 0.01% w/v to 5% w/v.

An embodiment of the present disclosure provides a nucleic acid amplification reagent gel, including the gel precursor provided by an embodiment of the present disclosure, and further including: one or more reagents, mixed in the gel precursor, needed for nucleic acid amplification, and a salt solution containing a first ion, where the first ion and the gel precursor are subjected to a cross-linking reaction to form a gel, and to embed the one or more reagents into the gel; where

the nucleic acid amplification reagent gel is configured that when a salt solution containing a second ion is added, the second ion and the gel are subjected to an ion exchange reaction to dissolve the gel, and to release the one or more reagents.

In one embodiment, the first ion is a divalent metal cation.

In one embodiment, the divalent metal cation is at least one of a Ca²⁺ ion and a Ba²⁺ ion.

In one embodiment, the second ion is a monovalent metal cation.

In one embodiment, the monovalent metal cation is at least one of a Na⁺ ion and a K⁺ ion.

In one embodiment, the salt solution containing the second ion is a sodium citrate solution containing the second ion, or an ethylenediaminetetraacetic acid disodium salt solution containing the second ion.

An embodiment of the present disclosure further provides a storage chip for a nucleic acid amplification reagent gel, including: a first storage cavity, a sample pool, interconnected with the first storage cavity, configured to hold an object to be detected, and a second storage cavity, interconnected with the first storage cavity, configured to store at least one of a gel dissolving agent and a buffer solution; and

where the first storage cavity stores the nucleic acid amplification reagent gel provided by the embodiment of the present disclosure.

An embodiment of the present disclosure provides a using method of a nucleic acid amplification reagent gel, including:

when one or more reagents needed for nucleic acid amplification are stored, mixing the gel precursor provided by the embodiment of the present disclosure containing one or more the reagents and a salt solution containing a first ion to allow the first ion and the gel precursor to be subjected to a cross-linking reaction to form a gel, and to embed the one or more reagents into the gel; and

when the one or more reagents are released, adding a salt solution containing a second ion into the gel to allow the second ion and the gel to be subjected to an ion exchange reaction to dissolve the gel, and to release the one or more reagents.

In one embodiment, the mixing the gel precursor containing the one or more reagents and the salt solution containing the first ion includes:

preparing the gel precursor;

adding the one or more reagents into the gel precursor; and

mixing the gel precursor and the salt solution containing the first ion.

In one embodiment, the adding the one or more reagents into the gel precursor includes:

preparing multiple reagents needed for nucleic acid amplification in advance and evenly mixing the multiple reagents; and

adding the multiple evenly mixed reagents into the gel precursor and conducting even mixing again.

In one embodiment, the adding the one or more reagents into the gel precursor includes:

preparing multiple reagents needed for nucleic acid amplification respectively; and

piercing the multiple reagents into different positions in the gel precursor respectively by means of a capillary tube or a microneedle.

In one embodiment, the preparing the gel precursor includes: preparing a sodium alginate aqueous solution.

In one embodiment, the preparing the gel precursor further includes: adding one or a combination of the following into the sodium alginate aqueous solution:

starch;

dextrin;

chitosan;

agarose; and

gelatin.

In one embodiment, the mixing the gel precursor and the salt solution containing the first ion includes: mixing the gel precursor and a salt solution containing a divalent metal ion.

In one embodiment, the mixing the gel precursor and the salt solution containing the divalent metal ion includes:

mixing the gel precursor and a salt solution containing at least one of a Ca²⁺ ion and a Ba²⁺ ion.

In one embodiment, the adding the salt solution containing the second ion into the gel includes:

adding a salt solution containing a monovalent metal ion into the gel.

In one embodiment, the adding the salt solution containing the monovalent metal ion into the gel includes:

adding a salt solution containing at least one of a Na⁺ ion and a K⁺ ion into the gel.

In one embodiment, the adding the salt solution containing at least one of the Na⁺ ion and the K⁺ ion into the gel includes:

adding a sodium citrate solution containing at least one of the Na⁺ ion and the K⁺ ion into the gel.

In one embodiment, each of the one or more reagents is includes one or a combination of the following:

a magnetic bead;

an enzyme;

a deoxynucleotide triphosphate; and

a Mg²⁺ ion.

In one embodiment, when the one or more reagents needed for nucleic acid amplification are stored, the using method further includes:

moving the gel into a chip, after the gel is formed; and

sealing and packaging the chip.

An embodiment of the present disclosure further provides a storage chip for a nucleic acid amplification reagent, including: a first storage cavity, a sample pool, interconnected with the first storage cavity, configured to hold an object to be detected, and a second storage cavity, interconnected with the first storage cavity, configured to store at least one of a gel dissolving agent and a buffer solution; and

the first storage cavity stores the one or more reagents in the using method provided by the embodiment of the present disclosure.

### BRIEF DESCRIPTION OF THE DRAWINGS

FIG. 1 is a flowchart of a using method of a nucleic acid amplification reagent gel provided by an embodiment of the present disclosure.

FIG. 2 is a flowchart of a specific using method of a nucleic acid amplification reagent gel provided by an embodiment of the present disclosure.

FIG. 3 is a flowchart of adding one or more reagents into a gel precursor provided by an embodiment of the present disclosure.

FIG. 4 is another flowchart of adding one or more reagents into a gel precursor provided by an embodiment of the present disclosure.

FIG. 5 is a flowchart of another specific using method of a nucleic acid amplification reagent gel provided by an embodiment of the present disclosure.

FIG. 6 is a storage chip for a nucleic acid amplification reagent gel provided by an embodiment of the present disclosure.

### DETAILED DESCRIPTION OF THE EMBODIMENTS

In order to make the object, technical solutions and advantages of embodiments of the present disclosure clearer, the technical solutions of the embodiments of the present disclosure will be clearly and completely described below in conjunction with the accompanying drawings of the embodiments of the present disclosure. Apparently, the described embodiments are only a part of the embodiments of the present disclosure, not all of the embodiments. Based on the described embodiments of the present disclosure, all other embodiments obtained by those of ordinary skill in the art without creative work shall fall within the protection scope of the present disclosure.

Unless otherwise defined, technical or scientific terms used in the present disclosure shall have the ordinary meanings understood by those ordinarily skilled in the art to which the present disclosure pertains. Words “first”, “second” and the like used in the present disclosure do not indicate any order, quantity or importance, but are only configured to distinguish different components. Words “comprise” or “include” and the like indicate that an element or item appearing before such the word covers listed elements or items appearing after the word and equivalents thereof, and does not exclude other elements or items. Words “connection” or “connected” and the like are not limited to physical or mechanical connection, but may include electrical connection, whether direct or indirect. “Upper”, “lower”, “left”, “right” and the like are only configured to represent relative position relationships, and the relative position relationships may also change accordingly after an absolute position of a described object is changed.

In order to keep the following descriptions of the embodiments of the present disclosure clear and concise, detailed descriptions of known functions and known components are omitted.

An embodiment of the present disclosure provides a gel precursor, including:

a sodium alginate main solution and at least one of the following components mixed in the sodium alginate main solution:

starch;

dextrin;

chitosan;

agarose; and

gelatin;

where the sodium alginate main solution has a mass concentration range from 0.01% w/v to 5% w/v.

The gel precursor provided by the embodiment of the present disclosure includes: the sodium alginate main solution and at least one of the following components mixed in the sodium alginate main solution: the starch, the dextrin, the chitosan, the agarose and the gelatin. Since the starch, the dextrin, the chitosan, the agarose and the gelatin contain hydrogen bonds, when they are added into the sodium alginate main solution, rapid forming may be realized when a gel is subsequently formed, stability is good, and long-time preservation is achieved. When a reagent needed for nucleic acid amplification is stored, the gel precursor containing the reagent and a salt solution containing a first ion are mixed to allow the first ion and the gel precursor to be subjected to a cross-linking reaction so as to form a gel, and the reagent is embedded into the gel. Since the gel is in a solid state or a semi-solid state, transportation is easy, and the reagent may still maintain good activity in the transportation process. When the reagent needs to be used, a salt solution containing a second ion is added into the gel to allow the second ion and the gel to be subjected to an ion exchange reaction, and the gel is dissolved to release the reagent for nucleic acid amplification, thereby relieving the problems that the preservation effect is not good and the reagent performance is lowered when the reagents needed for nucleic acid amplification are stored and transported in the related art.

An embodiment of the present disclosure further provides a nucleic acid amplification reagent gel, including the gel precursor provided by the embodiment of the present disclosure, and further including: one or more reagents needed for nucleic acid amplification and mixed in the gel precursor, and a salt solution containing a first ion. The first ion and the gel precursor are subjected to a cross-linking reaction to form a gel, and to embed the reagent needed for nucleic acid amplification into the gel. In some embodiments, the first ion is a divalent metal cation, for example, the divalent metal cation is a Ca²⁺ ion and/or a Ba²⁺ ion.

The nucleic acid amplification reagent gel is configured that when a salt solution containing a second ion is added, the second ion and the gel are subjected to an ion exchange reaction, and the gel is dissolved to release the reagent. In some embodiments, the second ion is a monovalent metal cation, for example, the monovalent metal cation is a Na⁺ ion and/or a K⁺ ion. In some embodiments, the salt solution containing the second ion is a sodium citrate solution containing a second ion, or an ethylenediaminetetraacetic acid disodium salt solution containing a second ion.

Based on the same inventive concept, an embodiment of the present disclosure further provides a storage chip for a nucleic acid amplification reagent gel, as shown in FIG. 6, including: a first storage cavity 1, a sample pool 3 interconnected with the first storage cavity 1, and a second storage cavity 2 interconnected with the first storage cavity 1. The sample pool 3 is configured to hold an object to be detected, e.g., to hold samples to be operated such as blood samples and cells. The second storage cavity 2 is configured to store a gel dissolving agent and/or a buffer solution. The first storage cavity 1 stores the nucleic acid amplification reagent gel provided by the embodiment of the present disclosure. The gel dissolving agent may be a salt solution containing a second ion in a using method provided by an embodiment of the present disclosure.

In some embodiments, the storage chip may further include a curved micro-channel 4 interconnected with the first storage cavity 1 and a detection and waste liquid storage chamber 5 interconnected with the other end of the micro-channel 4.

Referring to FIG. 1, an embodiment of the present disclosure provides a using method of a nucleic acid amplification reagent gel, including:

step 100, when one or more reagents needed for nucleic acid amplification are stored, mixing a first solution containing the one or more reagents and a salt solution containing a first ion to allow the first ion and the first solution to be subjected to a cross-linking reaction to form a gel, and to embed the one or more reagents into the gel; and

step 200, when the one or more reagents are released, adding a salt solution containing a second ion into the gel to allow the second ion and the gel to be subjected to an ion exchange reaction to dissolve the gel, and to release the one or more reagent.

In some embodiments, the first solution provided by the embodiment of the present disclosure is a gel precursor, including a sodium alginate main solution mixed with at least one of the following components: starch, dextrin, chitosan, agarose and gelatin. Since the starch, the dextrin, the chitosan, the agarose and the gelatin contain hydrogen bonds, when they are added into the sodium alginate main solution, rapid forming may be realized when a gel is subsequently formed, stability is good, and long-time preservation is achieved. When the reagent needed for nucleic acid amplification is stored, the gel precursor containing the reagent and the salt solution containing the first ion are mixed to allow the first ion and the gel precursor to be subjected to the cross-linking reaction to form the gel, and the reagent is embedded into the gel. Since the gel is in a solid state or a semi-solid state, transportation is easy, and the reagent may still maintain good activity in the transportation process. When the reagent needs to be used, the salt solution containing the second ion is added into the gel to allow the second ion and the gel to be subjected to the ion exchange reaction, and the gel is dissolved to release the reagent for nucleic acid amplification, thereby relieving the problems that the preservation effect is not good and the reagent performance is lowered when the reagents needed for nucleic acid amplification are stored and transported in the related art.

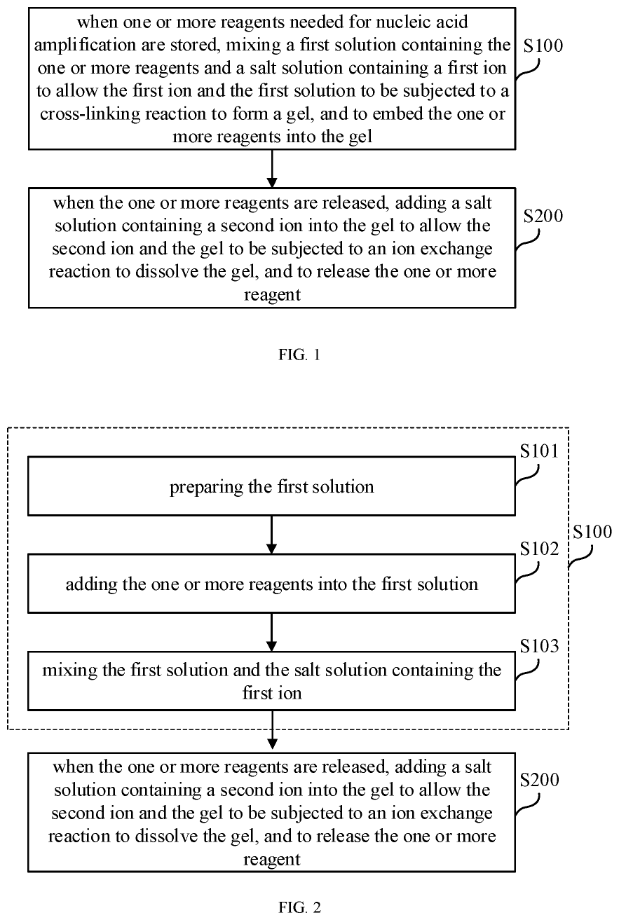

In some embodiments, referring to FIG. 2, the step S100 that the first solution containing the one or more reagents and the salt solution containing the first ion are mixed includes:

step S101, preparing the first solution. In some embodiments, the first solution is the gel precursor, and the step may be preparation of a sodium alginate aqueous solution. In some embodiments, a sodium alginate solute may be fully dissolved and evenly stirred to discharge bubbles, and the mass concentration of sodium alginate in the gel precursor specifically may range from 0.01% w/v to 5% w/v. In some embodiments, one or a combination of the following may be further added into the gel precursor: starch, dextrin, chitosan, agarose and gelatin. One or a combination of the starch, the dextrin, the chitosan, the agarose and the gelatin is added into the gel precursor, the viscosity of the gel precursor may be increased, and the solidification performance of the subsequently formed gel is enhanced. Where the gel precursor is further added with one or a combination of the starch, the dextrin, the chitosan, the agarose and the gelatin, a mixing ratio of the sodium alginate to these components may range from 50% to 100%;

step S102, adding the one or more reagents into the first solution. In some embodiments, the first solution is the gel precursor, and each of the one or more reagents should be substances that do not allow the gel precursor to be subjected to a reaction of degeneration such as the cross-linking reaction, and substances that do not react by inactivating or degenerating themselves in contact with the gel. The reagent specifically includes, but is not limited to, one or more of a magnetic bead, an enzyme, a deoxynucleotide triphosphate (dNTP) and a Mg²⁺ ion, and the concentration and adding ratio of the reagent may be determined according to a whole reaction system. For example, only the magnetic beads are stored alone in 0.05 g of gel with the mass volume fraction of 1%, where the storage amount of the magnetic beads is 10 μL. For another example, the enzymes, the dNTPs and the Mg²⁺ ions are stored in 0.1 g of gel with the mass volume fraction of 2% at the same time, where the storage amount of the enzymes ranges from 1 U/50 μL to 2 U/50 μL, the storage amount of the dNTPs is 0.2 mM, and the storage amount of the Mg²⁺ ions ranges from 1 mM to 4 mM. It needs to be noted that calculation of the concentrations of the above reagents needs to take the amount of water used in preparation of the gel precursor into account; and

step S103, mixing the first solution and the salt solution containing the first ion. In some embodiments, the first solution is the gel precursor, and the first ion may specifically be a divalent metal ion, that is, the step may be: mixing the gel precursor and the salt solution containing the divalent metal ion. In some embodiments, the first ion may further be at least one of a Ca²⁺ ion and a Ba²⁺ ion, and the step may further be: mixing the gel precursor and the salt solution containing at least one of the Ca²⁺ ion and the Ba²⁺ ion. Correspondingly, when the one or more reagents are released, the second ion may be a monovalent metal ion, and adding the salt solution containing the second ion into the gel may be: adding the salt solution containing the monovalent metal ion into the gel. Further, the second ion may be at least one of a Na⁺ ion and a K⁺ ion, that is, the step may further be: adding the salt solution containing at least one of the Na⁺ ion and the K⁺ ion into the gel. In the embodiment of the present disclosure, the first ion is the divalent metal ion, and the second ion is the monovalent metal ion. Since the monovalent metal ions usually have the more active chemical performance than the divalent metal ions, the salt solution containing the second ion may further be subjected to the ion exchange reaction with the gel, so that the gel is dissolved.

In some embodiments, when there are multiple reagents needed for nucleic acid amplification, the multiple reagents may be firstly mixed and then added into the gel precursor, or each reagent may be separately added into the gel precursor, which is illustrated through specific examples as follows.

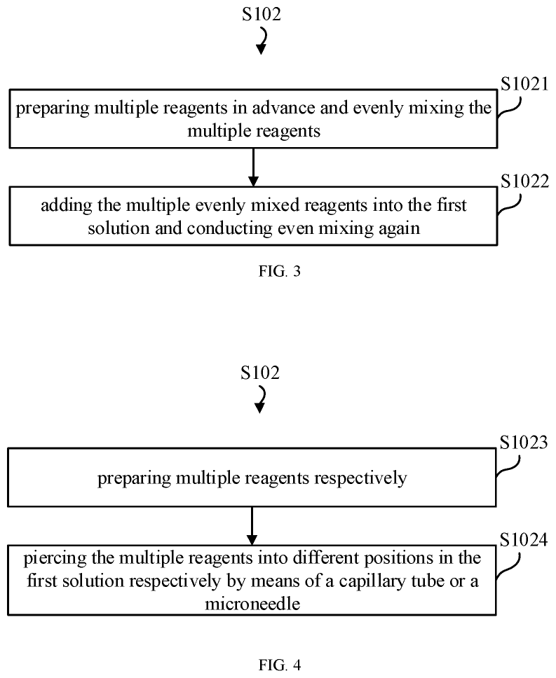

For example, referring to FIG. 3, the step S102 that the one or more reagents are added into the first solution includes:

step S1021, preparing multiple reagents in advance and evenly mixing the multiple reagents; and

step S1022, adding the multiple evenly mixed reagents into the first solution and conducting even mixing again.

In some embodiments, the first solution is the gel precursor. In the embodiment of the present disclosure, the multiple reagents are prepared in advance and evenly mixed, and then the multiple evenly mixed reagents are added into the gel precursor to be evenly mixed again, so that the step of adding the reagent into the gel precursor is simple, and the time is saved.

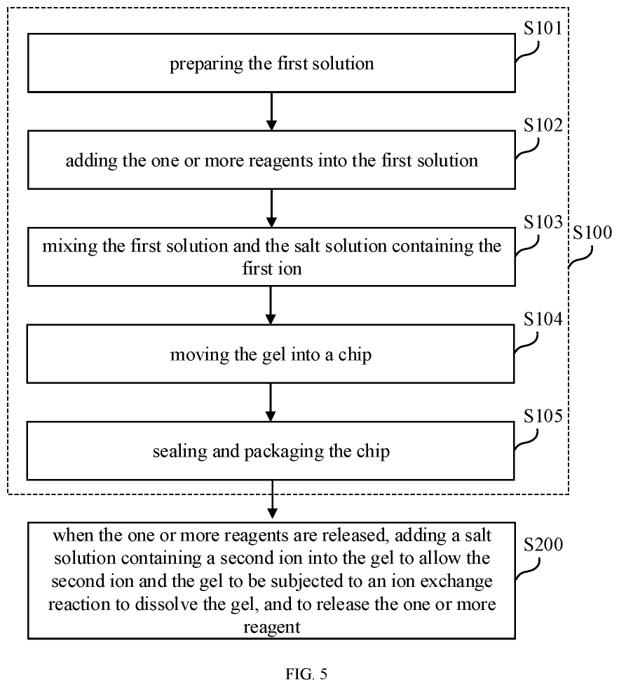

For another example, referring to FIG. 4, the step S102 that the one or more reagents are added into the first solution includes:

step S1023, preparing multiple reagents respectively; and

step S1024, piercing the multiple reagents into different positions in the first solution respectively by means of a capillary tube or a microneedle.

In some embodiments, the first solution is the gel precursor. In the embodiment of the present disclosure, the multiple reagents are prepared respectively, and then allowed to be pierced into the different positions in the gel precursor respectively by means of the capillary tube or the microneedle, so that the multiple reagents may not affect each other, and the activity of the reagents is prevented from being affected by mutual contact of the multiple reagents.

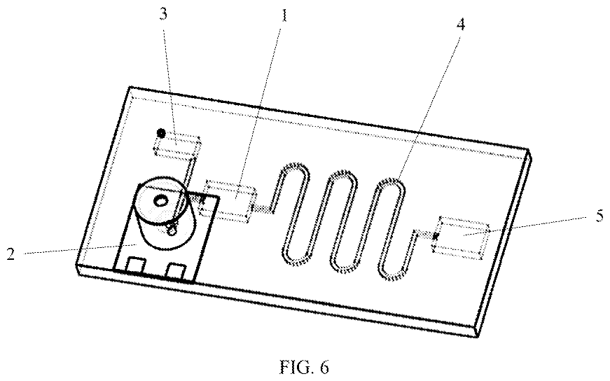

In some embodiments, referring to FIG. 5, when the one or more reagents needed for nucleic acid amplification is stored, the using method further includes:

step S104, moving the gel into a chip, after the gel is formed. In some embodiments, the gel may be, for example, moved manually, or may be moved by a mechanical arm, so that the solidified reagent gel is moved into a specific area of the used chip; and

step S105, sealing and packaging the chip. In some embodiments, sealing means that the specific area, containing the reagent gel, of the chip is sealed to isolate the area from external water and air before using. Specific measures include, but are not limited to, hot-pressing sealing, laser welding sealing, ultrasonic welding sealing, gluing, etc. Packaging means packaging the manufactured standby chip into a packaging bag in a light-avoiding closed mode to facilitate long-term storage and transportation.

In some embodiments, when the reagent is released, the salt solution containing the second ion specifically may be a sodium citrate solution, that is, the adding the salt solution containing at least one of a Na⁺ ion and a K⁺ ion into the gel may include: adding the sodium citrate solution containing at least one of the Na⁺ ion and the K⁺ ion into the gel. As an implementation, when the sodium alginate containing the reagent has the concentration of 1% and the mass of 0.05 g, after the sodium alginate forms calcium alginate gel, 0.1 mL of a 10% sodium citrate aqueous solution may be introduced to make full contact with the gel, the calcium alginate gel may be completely dissolved in 1 min, and the pre-stored reagent in the gel is released. Obviously, calculation of the concentrations of the used reagents needs to take the amount of water in the aqueous solution used for dissolving the gel into account. In the embodiment of the present disclosure, the adopted sample is blood which contains sodium citrate as an anticoagulant. When the blood sample makes contact with the gel containing the reagent, the sodium citrate may show the function of dissolving the gel at the same time.

In some embodiments, the salt solution containing the second ion may also specifically be an ethylenediaminetetraacetic acid disodium salt (EDTA-2Na) solution, the monovalent metal cation and the calcium alginate gel are subjected to ion exchange, the gel is completely dissolved, and the reagent, stored in the gel, needed for nucleic acid amplification is released.

Based on the same inventive concept, an embodiment of the present disclosure further provides a storage chip for a nucleic acid amplification reagent, as shown in FIG. 6, including: a first storage cavity 1, a sample pool 3 interconnected with the first storage cavity 1, and a second storage cavity 2 interconnected with the first storage cavity 1. The sample pool 3 is configured to hold an object to be detected, e.g., to hold samples to be operated such as blood samples and cells. The second storage cavity 2 is configured to store a gel dissolving agent and/or a buffer solution. The first storage cavity 1 stores the reagents of the using method provided by the embodiment of the present disclosure. The gel dissolving agent may specifically be the salt solution containing the second ion in the using method provided by the embodiment of the present disclosure.

In some embodiments, the storage chip may further include a curved micro-channel 4 interconnected with the first storage cavity 1 and a detection and waste liquid storage chamber 5 interconnected with the other end of the micro-channel 4.

The embodiments of the present disclosure have the following beneficial effects: the gel precursor includes: the sodium alginate main solution mixed with the following components: the starch, the dextrin, the chitosan, the agarose and the gelatin; since the starch, the dextrin, the chitosan, the agarose and the gelatin contain the hydrogen bonds, when they are added into the sodium alginate main solution, rapid forming may be achieved when the gel is subsequently formed, stability is good, and long-time preservation is achieved; when the reagent needed for nucleic acid amplification is stored, the gel precursor containing the reagent and the salt solution containing the first ion are mixed to allow the first ion and the gel precursor to be subjected to the cross-linking reaction to form the gel, and the reagent is embedded into the gel; since the gel is in the solid state or the semi-solid state, transportation is easy, and the reagent may still maintain good activity in the transportation process; and when the reagent needs to be used, the salt solution containing the second ion is added into the gel to allow the second ion and the gel to be subjected to the ion exchange reaction, and the gel is dissolved to release the reagent for nucleic acid amplification, thereby relieving the problems that the preservation effect is not good and the reagent performance is lowered when the reagents needed for nucleic acid amplification are stored and transported in the prior art.

Apparently, those skilled in the art can make various modifications and variations to the present disclosure without departing from the spirit and scope of the present disclosure. In this way, under the condition that these modifications and variations of the present disclosure fall within the scope of the claims of the present disclosure and their equivalent technologies, the present disclosure is also intended to include these modifications and variations.

What is claimed is:

1. A gel precursor, comprising: a sodium alginate main solution mixed with at least one of following components:
starch;
dextrin;
chitosan;
agarose; and
gelatin;
wherein the sodium alginate main solution has a mass concentration range from 0.01% w/v to 5% w/v.

2. A nucleic acid amplification reagent gel, comprising the gel precursor of claim 1, and further comprising:
one or more reagents, mixed in the gel precursor, needed for nucleic acid amplification, and
a salt solution containing a first ion,
wherein the first ion and the gel precursor are subjected to a cross-linking reaction to form a gel, and to embed the one or more reagents into the gel; and
the nucleic acid amplification reagent gel is configured that when a salt solution containing a second ion is added, the second ion and the gel are subjected to an ion exchange reaction to dissolve the gel, and to release the one or more reagents.

3. The nucleic acid amplification reagent gel of claim 2, wherein the first ion is a divalent metal cation.

4. The nucleic acid amplification reagent gel of claim 3, wherein the divalent metal cation is at least one of: a Ca²⁺ ion and a Ba²⁺ ion.

5. The nucleic acid amplification reagent gel of claim 2, wherein the second ion is a monovalent metal cation.

6. The nucleic acid amplification reagent gel of claim 5, wherein the monovalent metal cation is at least one of: a Na⁺ ion and a K⁺ ion.

7. The nucleic acid amplification reagent gel of claim 6, wherein:
the salt solution containing the second ion is a sodium citrate solution containing the second ion, or
the salt solution containing the second ion is an ethylenediaminetetraacetic acid disodium salt solution containing the second ion.

8. A storage chip for a nucleic acid amplification reagent gel, comprising:
a first storage cavity,
a sample pool, interconnected with the first storage cavity, configured to hold an object to be detected, and
a second storage cavity, interconnected with the first storage cavity, configured to store at least one of: a gel dissolving agent and a buffer solution;
wherein the first storage cavity stores the nucleic acid amplification reagent gel of claim 2.

9. A using method of a nucleic acid amplification reagent gel, comprising:
when one or more reagents needed for nucleic acid amplification are stored, mixing the gel precursor of claim 1 containing the one or more reagents and a salt solution containing a first ion to allow the first ion and the gel precursor to be subjected to a cross-linking reaction to form a gel, and to embed the one or more reagents into the gel; and
when the one or more reagents are released, adding a salt solution containing a second ion into the gel to allow the second ion and the gel to be subjected to an ion exchange reaction to dissolve the gel, and to release the one or more reagents.

10. The using method of claim 9, wherein the mixing the gel precursor containing the one or more reagents and the salt solution containing the first ion comprises:
preparing the gel precursor,
adding the one or more reagents into the gel precursor; and
mixing the gel precursor and the salt solution containing the first ion.

11. The using method of claim 10, wherein the adding the one or more reagents into the gel precursor comprises:
preparing multiple reagents needed for nucleic acid amplification in advance and evenly mixing the multiple reagents; and
adding the multiple evenly mixed reagents into the gel precursor and conducting even mixing again.

12. The using method of claim 10, wherein the adding the one or more reagents into the gel precursor comprises:
preparing multiple reagents needed for nucleic acid amplification respectively; and
piercing the multiple reagents into different positions in the gel precursor respectively by means of a capillary tube or a microneedle.

13. The using method of claim 10, wherein the preparing the gel precursor comprises: preparing a sodium alginate aqueous solution; and the preparing the gel precursor further comprises:
adding one or a combination of following into the sodium alginate aqueous solution:
starch;
dextrin;
chitosan;
agarose; and
gelatin.

14. The using method of claim 10, wherein the mixing the gel precursor and the salt solution containing the first ion comprises: mixing the gel precursor and a salt solution containing a divalent metal ion.

15. The using method of claim 14, wherein the mixing the gel precursor and the salt solution containing the divalent metal ion comprises:
mixing the gel precursor and a salt solution containing at least one of a Ca²⁺ ion and a Ba²⁺ ion.

16. The using method of claim 15, wherein the adding the salt solution containing the second ion into the gel comprises:
adding a salt solution containing a monovalent metal ion into the gel.

17. The using method of claim 16, wherein the adding the salt solution containing the monovalent metal ion into the gel comprises:
adding a salt solution containing at least one of a Na⁺ ion and a K⁺ ion into the gel.

18. The using method of claim 17, wherein the adding the salt solution containing at least one of the Na⁺ ion and the K⁺ ion into the gel comprises:
adding a sodium citrate solution containing at least one of the Na⁺ ion and the K⁺ ion into the gel.

19. The using method of claim 9, wherein each of the one or more reagents comprises one or a combination of following components:
a magnetic bead;
an enzyme;
a deoxynucleotide triphosphate; and
a Mg²⁺ ion.

20. The using method of claim 9, wherein when the one or more reagents needed for nucleic acid amplification are stored, the using method further comprises:
moving the gel into a chip, after the gel is formed; and
scaling and packaging the chip.
